(19) United States
(12) Patent Application Publication
(10) Pub. No.: US 2025/0127281 A1
(43) Pub. Date: Apr. 24, 2025

(54) DEVICE FOR STRETCHING A PATCH, NOTABLY A COSMETIC PATCH

(71) Applicant: L'Oreal, Paris (FR)

(72) Inventors: Franck Giron, CHEVILLY-LARUE (FR); Henri Samain, CHEVILLY-LARUE (FR)

(73) Assignee: L'Oreal, Paris (FR)

(21) Appl. No.: 18/722,294

(22) PCT Filed: Dec. 21, 2022
(86) PCT No.: PCT/EP2022/087337

§ 371 Date: Jun. 20, 2024

(30) Foreign Application Priority Data
Dec. 21, 2021 (FR) 2114173
Dec. 21, 2021 (FR) 2114183

(51) Int. Cl. A45D 44/00; A61K 8/02; A61Q 19/00

(52) U.S. Cl. CPC A45D 44/002; A61K 8/0208; A61Q 19/00; A61K 2800/87

(57) ABSTRACT

This device for stretching a patch intended to be applied to the skin, notably a cosmetic patch, comprises a patch applicator (2) provided with a support (4), a pusher (5) provided with a cavity (6) and with a mounting surface (7) for the patch (3) to be stretched, and a base portion (8) comprising a protuberance (9) which is provided with a receiving surface (10) for the support of the applicator and which can be fitted into the cavity (6) of the pusher such that, when the patch to be stretched is placed on the mounting surface (7) of the pusher and said pusher and the base portion are fitted together, the outer surface of the patch is stretched uniformly outwards and bears against the support (4) of the applicator.

The present invention relates to stretchable patches that can be placed over regions that are to be treated, for example to cosmetic patches for the skin.

The present invention relates more particularly to the devices for stretching such patches.

The present invention also relates to methods of cosmetic analysis through the placement of stretched-out patches on regions that are to be analyzed and/or treated.

The expression “cosmetic product” is understood to mean a product as defined in Council Directive 93/35/EEC of 14 Jun. 1993.

When the skin exhibits positive or negative reliefs, also referred to as bumps or hollows, such as spots, scars or wrinkles, these may lessen the esthetic look of the skin.

The cosmetic solutions for the skin are generally confined to make-up or patches, which merely conceal the skin coloration defects while leaving the relief visible.

Solutions for reducing positive reliefs have been envisioned. For example, it has been envisioned for parts exhibiting a convex face facing toward the skin to be affixed in order to flatten the positive reliefs and thus reduce the height thereof.

However, this solution requires the convex parts to be of great thickness, of the order of one millimeter, which means that they cannot be discreet. In addition, because the convex parts need to be rigid, the movements of the skin make the parts all the more visible as they are unable to conform correctly to the skin.

Additionally, this type of part does not adhere well and in a few minutes to a few hours a convex part naturally becomes detached. Adhesion may be improved by providing flanges on the convex parts, but that amounts to using a part that is very wide for a limited region in positive relief, making the convex part all the more visible to the detriment of its discreetness.

It is therefore an object of the present invention to overcome the above-mentioned disadvantages by proposing a device for applying patches that are thinner and less rigid and able to rectify cosmetic skin problems.

The subject of the present invention is a device for stretching a patch intended to be applied to the skin, notably a cosmetic patch, comprising a patch applicator provided with a support, a pusher comprising a cavity and a mounting surface for the patch that is to be stretched, and a base unit comprising a protuberance which is provided with a receiving surface for the support of the applicator and can be fitted into the cavity of the pusher, so that when the patch that is to be stretched is placed on the mounting surface of the pusher and said pusher and the base unit are fitted together, the exterior surface of said patch is stretched uniformly outward and comes to bear against the support of the applicator.

Specifically and surprisingly it has been found that a patch thus stretched and adhered to the skin will exert forces directed toward the center of the patch and that the skin reacts by sinking inward rather than by bulging outward, thus reducing or even eliminating the positive relief over which the patch is positioned.

Thus, the device according to the invention allows such an elastic patch to be stretched uniformly and applied to a region that is to be treated. The region is therefore treated with a patch that is thin, invisible and able to conform to the movements of the skin. Finally, for different applications, the patches sometimes need to be stretched to a greater or lesser extent, and this is rendered possible by having different sizes of support and/or of protuberance.

Advantageously, the support of the applicator has a circular shape. This is even better for obtaining a patch that has been stretched uniformly. Specifically, it is even better at keeping the stretched patch in a circular shape. Alternatively, it is still possible to conceive of other shapes for the patch in the stretched state.

Advantageously, the mounting surface of the pusher is formed by a transverse end face from which the cavity extends.

In one embodiment, the applicator further comprises a ring surround intended to be mounted inside a housing of the cavity of the pusher and dimensioned to encircle the support when the pusher and the base unit are in the fitted-together position, the encircling of the support by the ring surround keeping the stretched patch bearing against the support of the applicator.

In one embodiment, the ring surround is equipped with a patch-cutting member so that when the ring surround is encircling the support with the patch, the latter is automatically cut to the size of the ring surround.

Advantageously, the support of the applicator comprises a planar first face and a planar or rounded second face which is the opposite face to the planar first face, the first and second faces delimiting the thickness of the support, the first face being intended to be in contact with the receiving surface of the base unit, the second face being intended to accept the stretched patch.

Another subject of the invention is a method for stretching a patch intended to be applied to the skin, notably a cosmetic patch, comprising a patch stretching step in which the exterior surface of said patch is stretched uniformly outward, and a step of keeping said patch that is to be applied in the stretched state.

In one particular embodiment, the method further comprises a step of applying the patch in the stretched state to the region that is to be treated.

Another subject of the invention is a method for stretching and applying a patch, notably a cosmetic patch, comprising the following steps:
placing a film on the mounting surface of the pusher of a patch stretching device as defined hereinabove;
placing the support of the applicator on the receiving surface of the base unit of the device;
bringing the film to bear on the support of the applicator while simultaneously performing the step of stretching the patch that is formed by that part of the film that is bearing against the support, by fitting together the base unit and the pusher of the device;
separating the pusher from the base unit;
removing the applicator from the pusher, and
applying the stretched patch to the region that is to be treated.

In one embodiment, the patch stretching and application method further comprises a step of placing a ring surround of the applicator inside the housing of the cavity of the pusher prior to the step of placing the film, the ring surround being dimensioned to encircle the support during the patch stretching step, the encircling of the support by the ring surround keeping the stretched formed patch bearing against the support of the applicator.

Advantageously, the application step is performed by bringing the support against the region that is to be treated and by pushing the support in order to separate it from the ring surround.

Advantageously, the method further comprises a step of automatically cutting out the patch after the step of stretching the patch and before the step of separating the pusher from the base unit.

The invention also relates to a cosmetic kit comprising an elastic patch containing at least one cosmetic product and means for keeping the patch in a stretched position in which the exterior surface of said patch is stretched uniformly outward.

What is meant by an “elastic patch” is a cosmetic film capable, because of the material used and/or because of the sizing thereof, of deforming under the action of external stress, and of returning through elasticity at least partly to the initial shape that it had prior to defamation once the external stress ceases to be applied.

As a preference, the patch has two opposite faces that delimit its thickness, one of said faces being adhesive.

Moreover, cosmetic treatments such as creams constitute a so-called curative approach to the problem of reducing the reliefs of the skin.

Conversely, it is also advantageous to develop a preventive approach to these reliefs so as to detect and/or treat the regions that have reliefs before or at the moment that these start to appear.

Part of the preventive approach consists for example in applying protection against climatic factors such as ultraviolet light, dryness, cold or heat. For example, the use of sun cream is effective when there is no wish to obtain a pronounced tan.

However, other factors such as dust and nutrition are factors that are less easy to identify and it is far more difficult to prevent skin changes brought about by such factors.

In particular, the difficulty stems from the fact that it is difficult to apply products quasi uniformly to all of the skin or to a region of the skin.

In addition, a product that is effective is a product that contains a concentrated proportion of active ingredient, but this ingredient is sometimes uncomfortable if applied to extensive regions. It is also generally rather expensive. The user therefore reduces the frequency of use, the end result being correspondingly diminished.

Finally, it is a notable fact that the skin changes non-uniformly and an overall prevention treatment does not guarantee an overall result of the same nature and may even contribute to accentuate in the nonuniform nature of the skin.

Tools for the analysis or preventive treatment of the skin exist, but are expensive and require the assistance of a dermatologist or of a specialist.

It is therefore another object of the present invention to overcome the above-mentioned disadvantages by proposing a method for applying stretched patches to the skin in order to perform preventive analysis of the skin. Additionally, the present invention seeks to reduce the positive reliefs visible on the skin.

Another subject of the present invention is a cosmetic analysis method comprising the following steps:
applying to at least one region of the skin that is to be analyzed at least one patch that has been stretched uniformly outward; and
analyzing the at least one region that is to be analyzed by measuring the mechanical resistance after the step of applying the patch.

Specifically and surprisingly it has been found that a patch thus stretched and adhered to the skin will exert forces directed toward the center of the patch and that the skin reacts by sinking inward rather than by bulging outward, thus reducing or even eliminating the positive relief over which the patch is positioned.

In addition, a region of the skin can be analyzed by studying the reaction and the mechanical resistance of the skin. What is meant by “mechanical resistance of the skin” is its ability to mechanically resist the effect of the patch. This ability may be measured non-exhaustively by studying the contraction of the diameter of a circular region of skin or by studying an apparent diameter that contracts nonuniformly, for example when a circular region of skin becomes oval, or else by studying the contraction and/or the puckering and/or the inward sinking of the diameter of a circular region of skin.

The skin may be studied over the course of time or in comparison with other regions. Finally, a patch coated with a cosmetic treatment allows targeted treatment of certain regions using a patch that is thin, invisible and able to conform to the 15 movements of the skin.

In one embodiment, the step of analysis by measuring the mechanical resistance of the at least one region that is to be analyzed, is carried out by measuring the depth of the inward sinking of the region after the step of applying the patch.

In one embodiment, the step of analysis by measuring the mechanical resistance of the at least one region that is to be analyzed, is carried out by measuring the contracted diameter of the patch after the step of applying the patch.

Advantageously, the step of analysis by measuring the mechanical resistance of the at least one region that is to be analyzed is carried out by marking the region and/or of the patch with points or lines and/or with the use of natural points of reference.

Advantageously, the step of analysis by measuring the mechanical resistance of the at least one region that is to be analyzed is carried out by taking into consideration information regarding the climate and/or cosmetic treatments.

In one embodiment, the method further comprises the following steps:
identifying at least one region of potential change within the at least one analyzed region;
determining a cosmetic treatment to be applied to the at least one region of potential change on the basis of the mechanical resistance measurements taken during the analysis step; and
applying a cosmetic treatment product to the at least one region of potential change.

Advantageously, the steps of the method are repeated over the course of time in order to monitor the cosmetic analysis.

In one embodiment, in the application step, at least two stretched patches are applied over at least two distinct regions of the skin that is to be analyzed, in the analysis step, the mechanical resistance of the at least two regions to be analyzed is measured, and the step of identifying at least one region of potential change is carried out by making a comparison between at least two analyzed regions.

Advantageously, in the application step, at least two stretched patches are applied to at least two distinct regions of the skin that is to be analyzed, one of which regions is treated with a first cosmetic treatment product and another of which regions is treated with a second cosmetic treatment product different than the first cosmetic treatment product, or with no cosmetic treatment product, and in the step of determining a cosmetic treatment to be applied, it is determined which of the first, second or no cosmetic treatment product solutions is most suitable for applying to the analyzed regions of the skin.

Advantageously, the patch used comprises and/or is coated with cosmetic active ingredients.

In one particular embodiment, the step of applying a patch comprises the following steps:
placing a film on the mounting surface of the pusher of a patch stretching device, the device comprising a patch applicator provided with a support, a pusher comprising a cavity and a mounting surface for the patch that is to be stretched, and a base unit comprising a protuberance which is provided with a receiving surface for the support of the applicator and can be fitted into the cavity of the pusher, so that when the patch that is to be stretched is placed on the mounting surface of the pusher and said pusher and the base unit are fitted together, said patch is stretched and comes to bear against the support of the applicator;
placing the support of the applicator on the receiving surface of the base unit of the device;
bringing the film to bear on the support of the applicator while simultaneously performing the step of stretching the patch that is formed by that part of the film that is bearing against the support of the applicator, by fitting together the base unit and the pusher of the device;
separating the pusher from the base unit;
removing the applicator from the pusher, and
applying the stretched patch to the region that is to be analyzed.

Further aims, features and advantages of the invention will become apparent from reading the following description, which is given only by way of non-limiting example and with reference to the appended drawings, in which:

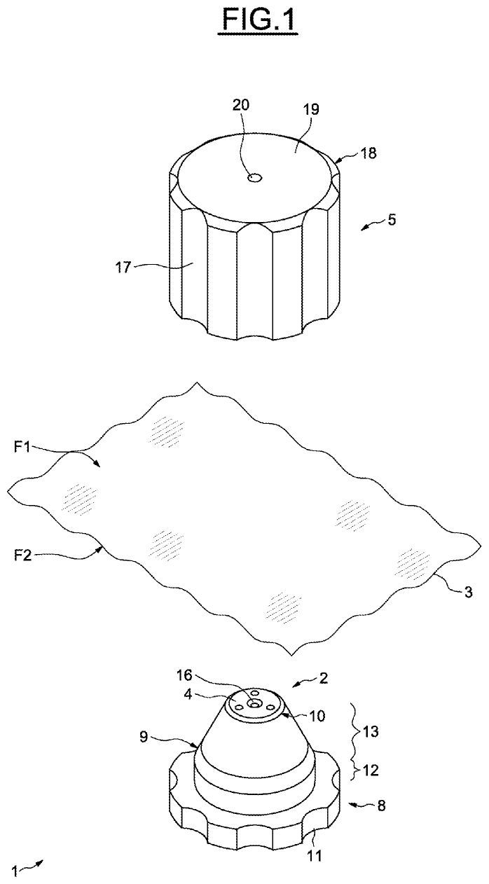

FIG. 1 is a schematic view of a patch stretching device in an open position according to one embodiment of the invention;

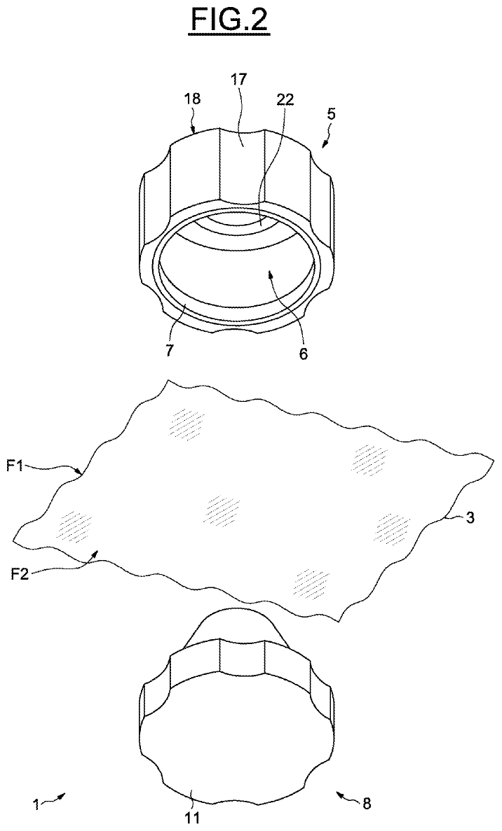

FIG. 2 is a view of the device of FIG. 1 in another orientation;

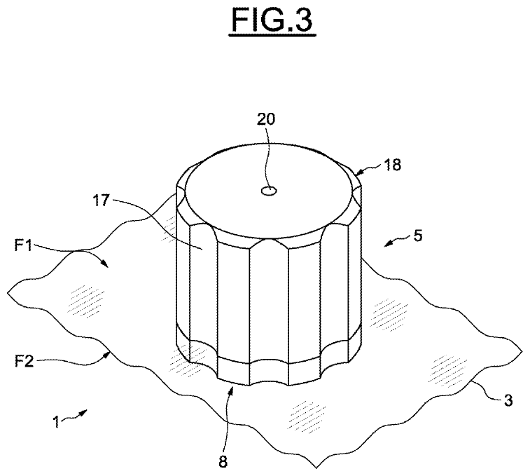

FIG. 3 is a schematic view of the device of FIGS. 1 and 2 in a closed position;

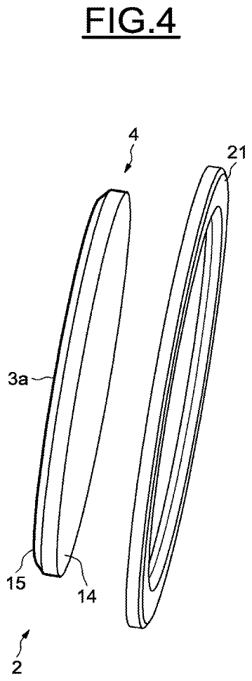

FIG. 4 is a view of a patch applicator of the device of FIGS. 1 to 3;

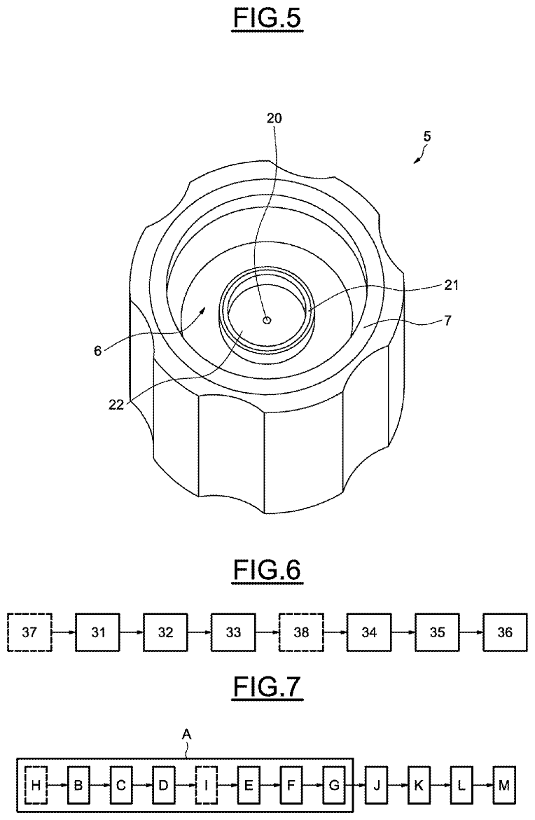

FIG. 5 is a view, from beneath, of a pusher of the device of FIGS. 1 to 3;

FIG. 6 illustrates the various steps of a patch stretching and application method according to one embodiment of the invention; and

FIG. 7 illustrates the various steps of a cosmetic analysis method using the application of a patch according to one embodiment of the invention.

In order to overcome the aforementioned disadvantages, the proposal is to use a stretchable film to form a patch, for example made from a polymer material. The material needs to have elastic properties such that it is able to exert a return force in the opposite direction to the force exerted for stretching it. Alternatively or in combination, the patch may be sized in such a way as to exhibit this elastic nature. The material used is thin, supple, and may be tinted, printed or translucent with a view to non-visibility, even when the skin is moving. In addition, it may include a coat of cosmetic product so that an active cosmetic ingredient can act on the skin when the patch is applied. In order to use the patch effectively, the patch needs to be stretched prior to being applied to the skin. Once applied, the patch tends to revert to its original shape, applying to the skin forces that are directed toward the center of the patch, the skin reacting by sinking inward. A non-stretched patch in the form of a film is, for example, a square with a side length of 45 to 100 millimeters, preferably a side length of around 50 millimeters. As a variant, the non-stretched patch in the form of a film may exhibit other shapes, for example other polygonal shapes, such as rectangular, triangular, etc., or even for example circular, oval, elliptical, etc.

The description below relates to a device for stretching such a patch, notably cosmetic patch.

FIGS. 1 and 2 depict a patch stretching device 1 which is in an open position. The device 1 is depicted in a position presumed to be vertical.

The device 1 comprises an applicator 2 for the patch that is to be stretched from a film 3 and is provided with a support 4 and a pusher 5 comprising an interior cavity 6 and a mounting surface 7 on which to mount the film 3.

The device 1 also comprises a base unit 8 comprising a protuberance 9 which is provided with a receiving surface 10 for the support 4 of the applicator and can be fitted into the cavity 6 of the pusher so that when the film 3, from which the patch that is to be stretched is formed, is placed on the mounting surface 7 of the pusher and said pusher 5 and the base unit 8 are fitted together, the exterior surface of said patch is stretched uniformly in a radial direction and comes to bear against the support 4 of the applicator. The radial direction of stretching of the patch is considered with reference to the axial direction of fitting-together of the pusher 5 and of the base unit 8. The entirety of the exterior surface of the patch is stretched radially. A closed position of the device in which position the pusher 5 and the base unit 8 are fitted together is illustrated in FIG. 3.

The pusher 5, the support 4 and the base unit 8 are for example made from a rigid synthetic material or from a metallic material. More specifically, the support 4 may be made of polytetrafluoroethylene, of polyoxymethylene or else of polyvinyl chloride.

The support 4 is a component separate from the base unit 8 and therefore from the receiving surface 10.

In the embodiment illustrated, the base unit 8 comprises a base 11 from which the protuberance 9 extends axially. The base 11 in this instance has a circular shape. Alternatively, it is possible to conceive of other shapes for the base 11.

The protuberance 9 is provided at its upper end with the receiving surface 10 that receives the support 4 of the applicator 2. The receiving surface 10 is planar. In the embodiment illustrated, the receiving surface 10 is circular.

In the embodiment illustrated, the protuberance 9 comprises a cylindrical portion 12 extending from the base 11, and a conical portion 13 prolonging the cylindrical portion 12 on the opposite side to the base 11 and delimiting the receiving surface 10 that receives the support 4 of the applicator.

In the embodiment illustrated, the support 4 of the applicator 2 is produced in the form of a disk the same size as the receiving surface 10 that receives the support 4.

As is notably visible in FIG. 4, the support 4 comprises a planar first face 14 and a second face 15 that is the opposite face to the first face 14. The first and second faces 14 and 15 delimit the thickness of the support 4. The first face 14 is intended to be in contact with the receiving surface 10 of the base unit (FIG. 1), the second face 15 being intended to accept the stretched patch 3a. The second face 15 may for example be planar or rounded. The support 4 may for example have a diameter of approximately 20 millimeters and a thickness of 1.5 millimeters.

Referring once again to FIG. 1, in the embodiment illustrated, the support 4 comprises at least one through-opening 16. The opening 16 extends through the thickness of the support 4. The opening 16 opens onto the first and second faces of the support. The opening 16 in this instance is centered on the support 4. The opening 16 allows the patch 3a to be detached easily at the time of application. The opening 16 also allows the support 4 to be correctly positioned on the receiving surface 10 of the base unit when this receiving surface 10 is equipped with a pin (not depicted) intended to be inserted into the opening 16 of the support 4.

The pusher 5 is a substantially hollow-cylindrical body of which the exterior surface 17 is intended to be grasped by the user and of which a transverse end face forms the mounting surface 7 for the film 3, from which surface the cavity 6 extends. In particular, the mounting surface 7 is an annular supporting surface for mounting the film 3 on the pusher 5. The pusher 5 comprises an annular skirt 18 delimiting the exterior surface 17. The free transverse end face of the skirt 18 forms the mounting surface 7. The pusher 5 also comprises an upper transverse wall 19 from which the skirt 18 extends. The skirt 18 of the pusher radially delimits the cavity 6. The transverse wall 19 axially delimits the cavity 6. The cavity 6 is open axially to the outside.

The pusher 5 is provided with an air-expulsion orifice 20 formed on the transverse wall 19 so as to allow the air to escape as the base unit 8 is being fitted together with the pusher 5.

As indicated previously, the stretchable cosmetic film 3 is intended to be placed on the mounting surface 7 of the pusher 5. The mounting surface 7 may for example be nonslip so as to prevent the patch 3a from slipping as it is being stretched. The film 3 has two opposite faces F1, F2 which delimit its thickness. The face F1 of the film 3, which is the face intended to form the surface of the patch 3a that is intended to come into contact with the skin, is placed against the mounting surface 7 of the pusher. This face F1 is, for example, adhesive. Alternatively, the face F1 is able not to comprise an adhesive layer. In that case, an adhesive may be applied afterwards either directly to the skin or to the patch 3a just before the patch is applied to the skin. The adhesive is, for example, a pressure-sensitive adhesive. The face F2 of the film 3 is the face intended to be in contact with the receiving surface 10 of the base unit.

As illustrated in FIGS. 4 and 5, in the embodiment illustrated, the applicator 2 further comprises a ring surround 21 which is intended to be mounted inside a housing 22 of the cavity 6 of the pusher 5. The housing 22 is situated at the bottom of the cavity 6. The housing 22 is situated at the level of the transverse wall 19 of the pusher.

The ring surround 21 is dimensioned to encircle the support 4 in the position in which the pusher 5 and the base unit 8 are fitted together, the encircling of the support 4 by the ring surround 21 keeping the stretched patch 3a formed from the film 3 pressed against the support 4 of the applicator 2. As the support 4 is being encircled by the surround 21, the latter comes radially into contact with the peripheral outer edge of the support.

The ring surround 21 is particularly useful when the properties of the material used for the patch 3a do not allow it to remain stretched unassisted and the patch 3a contracts rapidly. In such instances, it is effectively necessary to keep the patch 3a stretched before applying it to the skin, or quite simply for transporting it.

Conversely, when the properties of the material used for the patch 3a allow it to remain stretched unassisted for a few minutes before tending to revert to its original shape, the ring surround 21 is optional.

The ring surround 21 may for example be made from aluminum or from an aluminum-copper alloy, and its surface is obtained for example by micro-peening in order to improve its ability to keep the patch 3a under tension.

The ring surround 21 may comprise an edged cutting member for cutting the patch 3a so that when the ring surround 21 is encircling the support 4 with the patch 3a, the latter is automatically cut from the film 3 to the size of the ring surround 21.

FIG. 6 shows the various steps of the method for stretching and applying a patch 3a.

First of all, the step 31 of placing the film 3 on the mounting surface 7 of the pusher 5 of the device is performed. A, for example square, of stretchable film 3 with a side length in excess of 50 millimeters is prepared, and a protective layer protecting the face F1 is, if present, removed, and the face F1 of the film 3 which is the face intended to be in contact with the skin after the patch 3a has been formed is applied to the mounting surface 7 of the pusher.

Next, a step 32 of placing the support 4 of the applicator 2 on the receiving surface 10 of the base unit 8 of the device 1 is performed. Where present, a second protective layer situated on the other face F2 of the film 3 which is the face intended to be in contact with the support, is removed. Alternatively, the order of steps 31 and 32 may be reversed.

Next, the step 33 of radially stretching the patch 3a and of resting the stretched patch 3a on the support 4 of the applicator 2 is performed. In order to do this, the base unit 8 and the pusher 5 of the device are fitted together until such point as the base 11 of the base unit comes to bear axially against the mounting surface 7 of the pusher. During this step 33, the air-expulsion orifice 20 on the transverse face 18 must not be obstructed.

During this fitting-together, that part of the film 3 that is held against the mounting surface 7 of the pusher is deformed by the protuberance 9 of the base unit. In particular, that part of the film 3 that is pressed firmly against the support 4 mounted on the protuberance forms the patch 3a intended to be applied to the skin. The exterior surface of the patch 3a in the process of being formed is stretched radially outwards and uniformly over the entire periphery of this exterior surface. The patch 3a is stretched in a single plane.

Next, the pusher 5 and the base unit 8 are separated during the step 34, before the support 4 of the applicator, equipped with the stretched and formed patch 3a, is removed from the cavity 6 of the pusher 5 during step 35. Prior to being applied, the stretched patch 3a has a circular shape.

Finally, the stretched patch 3a is applied during step 36 to the region that is to be treated. The patch 3a is applied to the skin with the aid of pressure on the support 4.

As indicated previously, in the embodiment illustrated, the device comprises the ring surround 21. Thus, the method further comprises a step 37 of placing the ring surround 21 of the applicator 2 inside the housing 22 of the cavity of the pusher prior to the step 31 of placement of the film 3. As the base unit 8 and the pusher 5 of the device are being fitted together, the patch 3a that is in the process of being formed is stretched and held sandwiched between the ring surround 21 and the support 4. As indicated previously, the encircling of the support 4 by the ring surround keeps the patch 3 stretched in contact with the support 4 of the applicator 2.

The method further comprises a step 38 of automatically cutting out the patch 3a after the step 33 of stretching and before the step 34 of separating the pusher 5 from the base unit 8. The cutting step 38 is performed for example with the edged member (not depicted) of the ring surround 21.

The application step 36 is performed by bringing the support 4 against the region that is to be treated and by pushing the support 4 in order to separate it from the ring surround 21 and release the patch 3a onto the skin.

Finally, once the patch 3a is in place, it can be pressed down, notably along its edges, in order to make it adhere to the region that is to be treated. The patch 3a therefore applies a small amount of compression to the skin along these edges whereas the center of the patch 3a applies significant pressure to the skin, which sinks inward.

The patch 3a thus makes it possible to reduce relief effects, or apply cosmetic active ingredients, or else prevent future skin defects.

In this embodiment, the device 1 has a dual function of stretching the patch and of applying this patch.

Furthermore, it is possible, without departing from the scope of the invention, to envision means other than the device 1 for stretching out flat the exterior surface of the patch in a uniform manner.

Moreover, it is even possible to form the stretched patch and offer the patch to the users in this kept-stretched form. With this solution, users need only perform the step of applying the patch.

With this solution, in order to maintain the pre-load applied to the patch beforehand, the patch is associated with means for keeping said patch in its stretched position. These means may for example be the support and the ring surround as described hereinabove, or any other means able to perform this function. For example, these means may be a support on which the film is kept stretched through adhesive bonding.

The description hereinbelow relates to a preventive cosmetic analysis method using such a stretched patch and the various steps of which have been depicted in FIG. 7.

First of all, step A of applying at least one stretched patch to at least one region of the skin that is to be analyzed, is performed.

A region to be analyzed is, for example, a region of the face in which it is desirable to determine the short-term or medium-term evolution of the esthetic look of the skin. For example, it is useful to be able to foresee the appearance of spots. In order to do that, patches may be distributed across various locations on the face.

Application step A is performed for example using a patch-stretching device. A schematic embodiment of such a device is illustrated in FIGS. 1 and 2 in an open position and has already been described hereinabove. The device is depicted in a position presumed to be vertical. As an alternative, it is possible without departing from the scope of the invention to use a type of device other than the one described hereinbelow.

Referring once again to FIG. 7, during step A of application of a patch 3a, various steps are therefore performed in order to stretch and apply the patch 3a using the device.

First of all, the step B of placing the film 3 on the mounting surface 7 of the pusher 5 of the device is performed. A, for example, square of stretchable film 3 with a side length in excess of 50 millimeters is prepared, and a protective layer protecting the face F1 is, if present, removed, and the face F1 of the film 3 which is the face intended to be in contact with the skin after the patch 3a has been formed is applied to the mounting surface 7 of the pusher.

Next, a step C of placing the support 4 of the applicator 2 on the receiving surface 10 of the base unit 8 of the device 1 is performed. Where present, a second protective layer situated on the other face F2 of the film 3 which is the face intended to be in contact with the support, is removed. Alternatively, the order of steps B and C may be reversed.

Next, the step D of radially stretching the patch 3a and of resting the stretched patch 3a on the support 4 of the applicator 2 is performed. In order to do this, the base unit 8 and the pusher 5 of the device are fitted together until such point as the base 11 of the base unit comes to bear axially against the mounting surface 7 of the pusher. During this step D, the air-expulsion orifice 20 on the transverse face 18 must not be obstructed.

During this fitting-together, that part of the film 3 that is held against the mounting surface 7 of the pusher is deformed by the protuberance 9 of the base unit. In particular, that part of the film 3 that is pressed firmly against the support 4 mounted on the protuberance forms the patch 3a intended to be applied to the skin. The exterior surface of the patch 3a in the process of being formed is stretched radially outwards and uniformly over the entire periphery of this exterior surface. The patch 3a is stretched in a single plane.

Next, the pusher 5 and the base unit 8 are separated during the step E, before the support 4 of the applicator, equipped with the stretched and formed patch 3a, is removed from the cavity 6 of the pusher 5 during step F. Prior to being applied, the stretched patch 3a has a circular shape.

Finally, the stretched patch 3a is applied in step G to the region that is to be analyzed. The patch 3a is applied to the skin with the aid of pressure on the support 4.

As indicated previously, in the embodiment illustrated, the device comprises the ring surround 21. Thus, the method further comprises a step H of placing the ring surround 21 of the applicator 2 inside the housing 22 of the cavity of the pusher prior to the step B of placement of the film 3. As the base unit 8 and the pusher 5 of the device are being fitted together, the patch 3a that is in the process of being formed is stretched and held sandwiched between the ring surround 21 and the support 4. As indicated previously, the encircling of the support 4 by the ring surround keeps the patch 3 stretched in contact with the support 4 of the applicator 2.

The method further comprises a step I of automatically cutting out the patch 3a after the step D of stretching and before the step E of separating the pusher 5 from the base unit 8. The cutting step I is performed for example with the edged member (not depicted) of the ring surround 21.

The application step G is performed by bringing the support 4 against the region that is to be analyzed and/or treated and by pushing the support 4 in order to separate it from the ring surround 21 and release the patch 3a onto the skin.

Finally, once the patch 3a is in place, it can be pressed down, notably along its edges, in order to make it adhere to the region that is to be analyzed. The patch 3a therefore applies a small amount of compression to the skin along these edges whereas the center of the patch 3a applies significant pressure to the skin, which sinks inward.

The patch 3a thus makes it possible to reduce relief effects, or apply cosmetic active ingredients, or else prevent future skin defects.

In this embodiment, the device 1 has a dual function of stretching the patch and of applying this patch.

Furthermore, it is possible, without departing from the scope of the invention, to envision means other than the device 1 for stretching out flat the exterior surface of the patch in a uniform manner.

Moreover, it is even possible to form the stretched patch and offer the patch to the users in this kept-stretched form. With this solution, users need only perform the step of applying the patch.

With this solution, in order to maintain the pre-load applied to the patch beforehand, the patch is associated with means for keeping said patch in its stretched position. These means may for example be the support and the ring surround as described hereinabove, or other means able to perform this function. For example, these means may be a support on which the film is kept stretched through adhesive bonding.

In this last scenario of preventing future skin defects, there is next performed a step J of analysis by measuring the mechanical resistance of the skin in the region or regions where a patch 3a has been applied.

A mechanical-resistance analysis is performed for example by measuring the depth of the inward sinking of the region where a patch 3a has been applied. Alternatively, or in addition, the mechanical-resistance analysis is performed by measuring the contract diameter of the patch 3a when this patch is applied or once the patch 3a has been removed, if its contracted diameter can be identified by a mark. The mechanical-resistance measurements are performed for example manually using a graduated tool. Alternatively, the measurements may be taken using an optical sensor such as a camera or a laser profilometer, the measurement sought being deduced from the images acquired by the optical sensor.

The measurements, notably the measurement of the depth to which the skin has sunk inward, may be taken while the patch 3a is still in place. Alternatively, the measurement may be taken after the patch has been removed, it being possible for removal to occur several hours after application.

To facilitate measurement, markings may be used and drawn directly onto the skin or onto the patch. By way of example, a grid pattern of lines or of dots makes it easier to notice a sinking of the skin, the grid pattern becoming deformed as a result of the deformation of the surface of the skin upon application of the patch 3a. In instances in which measurements are taken by an optical sensor, an automatic comparison of the size of the grid pattern before and after application is made in order to deduce the sought-after measurement.

The measurements taken can then be compared against a first pre-established database in order to draw conclusions regarding the potential changes to certain regions. Additionally, the measurements taken may be weighted with coefficients dependent on the climate and/or on other factors that cause the measurements to vary. For example, certain cosmetic treatments applied to the skin may be taken into consideration. The coefficients that are dependent on the climate and/or on other factors that cause the measurements to vary are pre-established coefficients stored in the first database.

After the analysis step J, it is then possible to perform a step K of identifying one or more regions of potential change from among the region or regions analyzed following application of a patch 3a. These, for example, are regions in which the measurements diverge from skin measurements referred to as standard. Alternatively, an identified region corresponds to a region of skin which, in comparison with other regions of skin to which a patch 3a has been applied, displays a different mechanical resistance. Regions of potential change are therefore identified even before symptomatic reliefs appear on the skin.

Using a second database that links mechanical-resistance measurements to esthetic outcomes, a system then determines, during step L, a cosmetic treatment that is to be applied in the next step M. This may be an active ingredient to be ingested or to be spread on the skin. The active ingredient may also be coated onto future patches to be applied, or else the patch itself may contain such cosmetic active ingredients right from the time of manufacture. In one embodiment, the first and second databases may also be combined into the one same single database.

These steps A to M may be repeated as many times as necessary, notably over the course of time, in order to monitor changes to the skin. For example, a patch 3a is applied to the same region of skin every day, and mechanical-resistance measurements are taken for this region every day over the course of several months so as to refine the analysis and monitor changes to the skin as well as monitoring the effectiveness of any cosmetic product that might be involved. The method thus makes it possible to adapt the cosmetic product to be applied.

The invention finally allows a user to produce patches at home, apply them themselves and obtain an indication regarding the changes to their skin and the cosmetic products that need to be applied in order to remedy future esthetic defects. A specialist, such as a dermatologist, may also monitor the process remotely if need be.

In one embodiment, patches 3a are applied to several points on the cheeks. The patches are equipped with intersecting lines. It is then detected that the depth of inward sinking on one of the patches is lower than on the other patches. The other patches 3a are removed. Several hours later, a new measurement of the depth of inward sinking is taken on the remaining patch. These measurements are interpreted by a system or by a dermatologist which or who from them draws information regarding the changes to the skin. If the later depth of inward sinking is markedly lower than the earlier depth of inward sinking, the imminent appearance of a spot, currently not visible, can be deduced. Thus, that region can be preventively treated.

The operator may follow the same protocol on subsequent days.

1. A device for stretching a patch intended to be applied to the skin, notably a cosmetic patch, characterized in that it comprises a patch applicator (2) provided with a support (4), a pusher (5) provided with a cavity (6) and a mounting surface (7) for the patch that is to be stretched, and a base unit (8) comprising a protuberance (9) which is provided with a receiving surface (10) for the support of the applicator and can be fitted into the cavity (6) of the pusher, so that when the patch that is to be stretched is placed on the mounting surface (7) of the pusher and said pusher and the base unit are fitted together, the exterior surface of said patch is stretched uniformly outward and comes to bear against the support (4) of the applicator.

2. The device as claimed in claim 1, wherein the support (4) of the applicator (2) has a circular shape.

3. The device as claimed in claim 1, wherein the mounting surface (7) of the pusher (5) is formed by a transverse end face from which the cavity (6) extends.

4. The device as claimed in claim 1, wherein the applicator (2) further comprises a ring surround (21) intended to be mounted inside a housing (22) of the cavity (6) of the pusher (5) and dimensioned to encircle the support (4) when the pusher and the base unit (8) are in the fitted-together position, the encircling of the support (4) by the ring surround (21) keeping the stretched patch bearing against the support of the applicator.

5. The device as claimed in claim 4, wherein the ring surround (21) is equipped with a patch-cutting member so that when the ring surround (21) is encircling the support (4) with the patch, the latter is automatically cut to the size of the ring surround (21).

6. The device as claimed in claim 1, wherein the support (4) of the applicator comprises a planar first face (14) and a planar or rounded second face (15) which is the opposite face to the planar first face, the first and second faces delimiting the thickness of the support (4), the first face (14) being intended to be in contact with the receiving surface (10) of the base unit (8), the second face (15) being intended to accept the stretched patch.

7. A method for stretching a patch intended to be applied to the skin, notably a cosmetic patch, characterized in that it comprises a patch stretching step in which the exterior surface of said patch is stretched uniformly outward, a step of keeping said patch that is to be applied in the stretched state, a step of applying the patch in the stretched state to the region that is to be treated, the method further comprising the following steps:
placing a film (3) (step 31) on the mounting surface (7) of the pusher (5) of a stretching device as claimed in any one of claims 1 to 6;
placing the support (4) of the applicator on the receiving surface (10) of the base unit (8) of the device (step 32);
bringing the film (3) to bear on the support (4) of the applicator (2) while simultaneously performing the step of stretching the patch that is formed by that part of the film (3) that is bearing against the support, by fitting together the base unit (8) and the pusher (5) of the device (step 33);
separating the pusher from the base unit (step 34);
removing the applicator from the pusher (step 35), and
applying the stretched patch to the region that is to be treated (step 36).

8. The method as claimed in claim 7, further comprising a step of placing a ring surround (21) of the applicator inside the housing of the cavity of the pusher (step 37) prior to the step of placing the film, the ring surround being dimensioned to encircle the support (4) during the patch stretching step, the encircling of the support by the ring surround keeping the stretched formed patch bearing against the support of the applicator.

9. The method as claimed in claim 8, wherein the application step is performed by bringing the support (4) against the region that is to be treated and by pushing the support in order to separate it from the ring surround (21).

10. The method as claimed in claim 7, further comprising a step of automatically cutting out the patch (step 38) after the step of stretching the patch and before the step of separating the pusher (5) from the base unit (8).
